(19) United States
(12) Patent Application Publication
(10) Pub. No.: US 2025/0069706 A1
(43) Pub. Date: Feb. 27, 2025

(54) TCR-REPERTOIRE FUNCTIONAL UNITS

(71) Applicant: THE BOARD OF REGENTS OF THE UNIVERSITY OF TEXAS SYSTEM, Austin, TX (US)

(72) Inventor: Bo LI, Austin, TX (US)

(73) Assignee: THE BOARD OF REGENTS OF THE UNIVERSITY OF TEXAS SYSTEM, Austin, TX (US)

(21) Appl. No.: 18/729,385

(22) PCT Filed: Jan. 30, 2023
(86) PCT No.: PCT/US2023/061531

§ 371 Date: Jul. 16, 2024

Related U.S. Application Data
(60) Provisional application No. 63/267,369, filed on Jan. 31, 2022.

(51) Int. Cl. G16B 40/30; G16B 20/20

(52) U.S. Cl. CPC G16B 40/30; G16B 20/20

(57) ABSTRACT

A novel framework to transform a T-cell receptor (TCR) repertoire sample into a fixed-length vector. Short peptide sequences with different lengths in each TCR may be encoded into a numeric vector with fixed dimensions. A large amount of existing TCRs from healthy individuals may be pooled to generate a distribution of the encoding vector in a high-dimensional Euclidean space. Unsupervised clustering may be performed on the “points” in this space (each point is a TCR) to group them into antigen-specific clusters. The centroid of each cluster may be defined as a repertoire functional unit (“RFU”). For a new TCR repertoire sample, each TCR may be assigned to its most similar RFU group, and the RFU counts may be normalized by the number of sequences in the repertoire. The output data may be a fixed-length RFU vector, with each number representing the relative abundance of the given RFU in the repertoire.

### CROSS-REFERENCE TO RELATED APPLICATIONS

This application claims benefit of priority under 35 U.S.C. § 119 (e) of U.S. Provisional Application No. 63/267,369, filed Jan. 31, 2022. The disclosure of this application is considered part of and is herein incorporated by reference in the disclosure of this application in its entirety.

### TECHNICAL FIELD

The present disclosure generally relates to immune-repertoire based disease diagnosis technology, and more particularly to a novel system and method for efficiently grouping similar T cell receptor (TCR) sequences and diagnosing a patient with a disease and determining his/her disease status with a peripheral blood TCR repertoire.

### BACKGROUND

Adaptive immune repertoire is an important regulator of diverse human diseases, and over 10,000 TCR repertoire sequencing (TCR-seq) samples have been generated in the recent years. However, interpretation of TCR data has been hindered by the scarcity of known antigen-specificities. Recent studies demonstrated that similarity in the TCR hypervariable complementarity-determining region 3 (CDR3) implicates structural resemblance for antigen recognition. Therefore, clustering of similar CDR3s has become an important way to identify antigen-specific receptors.

### SUMMARY

Methods, systems, and apparati for transforming a T-cell receptor (TCR) repertoire sample into a fixed-length vector. Short peptide sequences with different lengths in each TCR may be encoded into a numeric vector with fixed dimensions. A large amount of existing TCRs from healthy individuals may be pooled to generate a distribution of the encoding vector in a high-dimensional Euclidean space. Unsupervised clustering may be performed on the “points” in this space (each point is a TCR) to group them into antigen-specific clusters. The centroid of each cluster may be defined as a repertoire functional unit (“RFU”). For a new TCR repertoire sample, each TCR may be assigned to its most similar RFU group, and the RFU counts may be normalized by the number of sequences in the repertoire. The output data may be a fixed-length RFU vector, with each number representing the relative abundance of the given RFU in the repertoire.

### BRIEF DESCRIPTION OF THE DRAWINGS

The drawings described below are for illustration purposes only. The drawings are not intended to limit the scope of the present disclosure.

FIG. 1 is a diagram of a system, according to some embodiments of the present disclosure;

FIG. 2 is a block diagram illustrating components for performing the methods described herein, according to some embodiments of the present disclosure;

FIG. 3 is a flowchart illustrating the workflow of numeric encoding framework used in the repertoire functional unit (RFU) process, according to some embodiments of the present disclosure;

FIG. 4 is a flowchart illustrating a geometric isometry based antigen-specific TCR alignment (GIANA) process, according to some embodiments of the present disclosure;

FIGS. 5A-5B are diagrams illustrating repertoire visualization using the trimer numeric encoding of TCRs, according to some embodiments of the present disclosure;

FIGS. 6A-6C are charts illustrating how the RFU process differentiates CD4 and CD8 T-cell repertoires, according to some embodiments of the present disclosure; and

FIGS. 7A-7B are charts illustrating how the RFU process differentiates cancer patients from healthy controls (“HCs”) and COVID-19 patients, according to some embodiments of the present disclosure.

### DETAILED DESCRIPTION

A number of conventional studies have applied TCR clustering to investigate antigen-specific T cell responses during disease progression or immunotherapy treatments. It is speculated that integrating a large number of TCR-seq samples from multiple studies will result in more insights into immune-disease interactions, and create novel opportunities for prognosis and diagnosis. Nonetheless, high clustering specificity requires pairwise Smith-Waterman alignment on both the CDR3 sequences and the TCR variable gene (TRBV) alleles, which has quadratic computational complexity that usually cannot scale up to the scale of TCR repertoire samples (≥100K sequences). Motif-based clustering achieves higher speed, but has much lower specificity. Therefore, none of the existing TCR clustering methods are suitable to analyze large cohorts of TCR-seq samples.

Unsupervised TCR clustering is a fundamental analysis of immune repertoire data. In the ideal scenario, all TCRs specific to the same epitope should be included in the same cluster. However, this is not feasible for sequence similarity or motif based clustering approach, due to the putative diversity in TCR sequences of shared specificity. Such diversity is caused by the distinct docking strategies of T cell receptors. For example, TCRs specific to the influenza GIL epitope usually contain the classic RSS/RSA motif in the CDR3 region, yet a related study reported that the LGGW motif also elicits strong binding to GIL from a different direction. Such structural variation cannot be captured by simple Smith-Waterman alignment, or motif grouping. Consequently, CDR3s with dissimilar motifs will be fragmented into smaller clusters despite their shared specificity, which is a common limitation to the current methods.

To address this challenge, a novel framework was developed to transform a TCR repertoire sample into a fixed-length “gene-expression-like” vector. First, each TCR sequence in the TCR repertoire may be numerically encoded. More specially, short peptide sequences with different lengths may be encoded into a numeric vector with fixed dimensions. Second, a large amount of existing TCRs from healthy individuals may be pooled to generate a distribution of the encoding vector in a high-dimensional Euclidean space. Unsupervised clustering may be performed on the “points” in this space (each point is a TCR) to group them into antigen-specific clusters. The centroid of each cluster may be defined as a Repertoire Functional Unit (“RFU”). For a new TCR repertoire sample, each TCR may be assigned to its most similar RFU group, and the RFU counts may be normalized by the number of sequences in the repertoire. The output data may be a fixed-length RFU vector, with each number representing the relative abundance of the given RFU in the repertoire.

The present disclosure will now be described more fully hereinafter with reference to the accompanying drawings, which form a part hereof, and which show, by way of non-limiting illustration, certain examples. Subject matter may, however, be described in a variety of different forms and, therefore, covered or claimed subject matter is intended to be construed as not being limited to any examples set forth herein. Among other things, subject matter may be described as methods, devices, components, or systems. Accordingly, examples may take the form of hardware, software, firmware or any combination thereof (other than software per se). The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, may be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession may be executed substantially concurrently or the blocks may sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5th generation (2G, 3G, 4G or 5G) cellular technology, Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

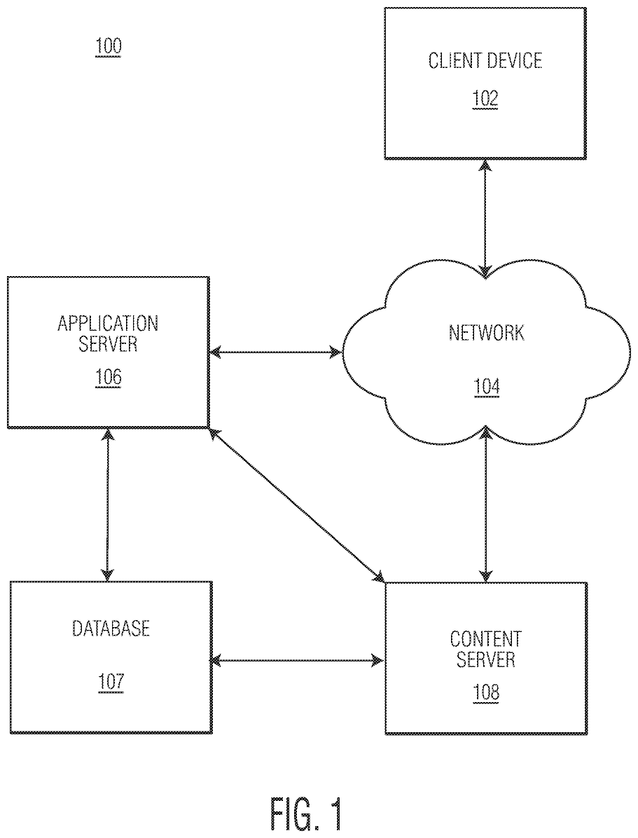

Referring now to FIG. 1, a system 100 is shown. FIG. 1 illustrates components of a general environment in which the systems and methods discussed herein may be practiced. Not all the components may be required to practice the disclosure, and variations in the arrangement and type of the components may be made without departing from the spirit or scope of the disclosure.

The system 100 of FIG. 1 includes network 104, which as discussed above, may include, but is not limited to, a wireless network, a local area network (LAN), wide area network (WAN), the Internet, or a combination thereof.

The network 104 may be connected, for example, to one or more client devices 102, an application server 106, a content server 108, and a database 107 and their components with another network or device. The network 104 may be configured as a variety of wireless sub-networks that may further overlay stand-alone ad-hoc networks, and the like, to provide an infrastructure-oriented connection for the one or more client devices 102, the application server 106, the content server 108, and the database 107. The network 104 may be configured to employ any form of computer readable media or network for communicating information from one electronic device to another.

The one or more client devices 102 may, for example, include a desktop computer or a portable device, such as a cellular telephone, a smart phone, a display pager, a radio frequency (RF) device, an infrared (IR) device, a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

The one or more client devices 102 may also include at least one client application that is configured to receive content from another computing device. The one or more client devices 102 may communicate over the network 104 with other devices or servers, and such communications may include sending and/or receiving messages, generating and providing TCR data, searching for, viewing and/or sharing TCR data, or any of a variety of other forms of communications. The one or more client devices 102 may be capable of processing or storing signals, such as in memory as physical memory states, and may, therefore, operate as a server

The application server 106 and the content server 108 may include one or more devices that are configured to provide and/or generate any type or form of content via a network to another device. Devices that may operate as the application server 106 and/or the content server 108 may include personal computers, desktop computers, multiprocessor systems, microprocessor-based or programmable consumer electronics, network PCs, servers, and the like. The application server 106 and the content server 108 may store various types of data related to the content and services provided by each device in the database 107.

Users (e.g., patients, doctors, technicians, and the like) may be able to access services provided by the application server 106 and the content server 108. This may include, for example, application servers, authentication servers, search servers, exchange servers, via the network 104 using the one or more client devices 102. Thus, the application server 106, for example, may store various types of applications and application related information including application data and user profile information.

Although FIG. 1 illustrates the application server 106 and the content server 108 as single computing devices, respectively, the disclosure is not so limited. For example, one or more functions of the application server 106 and the content server 108 may be distributed across one or more distinct computing devices. In another example, the application server 106 and the content server 108 may be integrated into a single computing device without departing from the scope of the present disclosure.

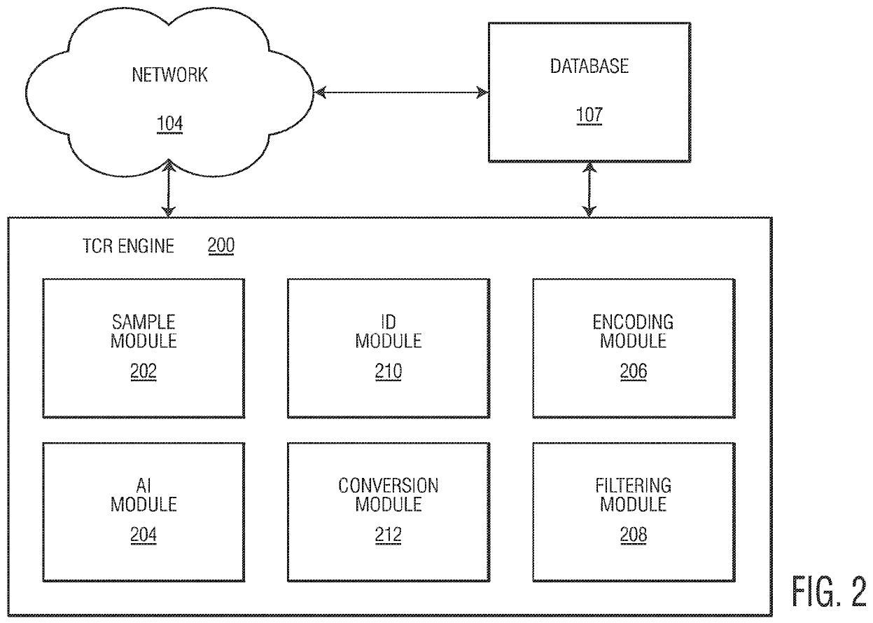

Referring now to FIG. 2, a block diagram illustrating components for performing the methods described herein is shown. FIG. 2 includes a TCR engine 200, the network 104, and the database 107. The TCR engine 200 may be a special purpose machine or processor and may be hosted by one or more of the application server 106, the content server 108, a web server, a third party server, a user's computing device, and the like.

In an example, the TCR engine 200 may be a conventional personal computer, and the methods described below may be performed using a single thread on a CPU. In another example, when clustering reference data of 10 million sequences, the TCR engine 200 may be a high-performance computing (HPC) super cluster (e.g., with 128G memory allocation and 8 CPU nodes).

The TCR engine 200 may be a stand-alone application that executes on a device (e.g., a user device or system/web-connected server/device). In another example, the TCR engine 200 may function as an application installed on the device and/or a web-based application accessed by the device over a network. The TCR engine 200 may be installed as an augmenting script, program or application (e.g., a plug-in or extension) to another application, such as, for example, a health care application that aggregates and shares patient related data.

The database 107 may be any type of database or memory, and may be associated with a server on a network (e.g., the application server 106 and the content server 108) or a user's device (e.g., the one or more client devices 102). The database 107 may include a dataset of data and metadata associated with local and/or network information related to users, services, applications, content and the like. Such information may be stored and indexed in the database 107 independently and/or as a linked or associated dataset. As discussed herein, it should be understood that the data (and metadata) in the database 107 can be any type of information and type, whether known or to be known, without departing from the scope of the present disclosure.

The database 107 may store data for users (e.g., user data. The stored user data may include, for example, information associated with reference TCR-seq data, a patient's cancer diagnosis, patient's chromosomal information, patient's DNA information, patient's blood information, patient demographic information, patient biographic information, and the like, or some combination thereof.

The data (and metadata) in the database 107 may be any type of information related to TCR-seq data, a patient, doctor, content, a device, an application, a service provider, a content provider, whether known or to be known, without departing from the scope of the present disclosure.

The data stored in the database 107 may be encrypted, for example, using a 256-bit encryption, such that the data is private and controlled according to Health Insurance Portability and Accountability Act of 1996 (HIPPA).

The database 107 may store and index the information as linked set of data and metadata, where the data and metadata relationship can be stored as the n-dimensional vector. Such storage can be realized through any known or to be known vector or array storage, including, but not limited to, a hash tree, queue, stack, VList, or any other type of known or to be known dynamic memory allocation technique or technology. It should be understood that any known or to be known computational analysis technique or algorithm, such as, but not limited to, cluster analysis, data mining, Bayesian network analysis, Hidden Markov models, artificial neural network analysis, logical model and/or tree analysis, and the like, and be applied to determine, derive or otherwise identify vector information for patients and/or health care providers.

As discussed above with reference to FIG. 1, the network 104 may be any type of network such as, but not limited to, a wireless network, a local area network (LAN), wide area network (WAN), the Internet, or a combination thereof. The network 104 may facilitate connectivity of the TCR engine 200 and the database 107 of stored resources. Indeed, as illustrated in FIG. 2, the TCR engine 200 and the database 107 may be directly connected by any known or to be known method of connecting and/or enabling communication between such devices and resources.

The principal processor, server, or combination of devices that include hardware programmed in accordance with the special purpose functions herein may be referred to for convenience as TCR engine 200. The TCR engine 200 may include a sample module 202, an AI module 204, an encoding module 206, a filtering module 208, an identification (ID) module 210, and a conversion module 212. The engine(s) and modules discussed herein are non-exhaustive, as additional or fewer engines and/or modules (or sub-modules) may be applicable to the examples of the systems and methods discussed. The operations, configurations and functionalities of each module, and their role within examples of the present disclosure are discussed below.

The principles described herein may be embodied in many different forms. T cells reactive to antigens are central mediators of immunity against various diseases and key targets of immunotherapies, yet as most disease antigens are unknown, experimental detection of disease-associated T cells remains difficult. The recent development of deep immune repertoire sequencing (TCR-seq) technology has placed an additional emphasis on the identification of such T cells, as it may open new opportunities for non-invasive clinical diagnosis, prognosis and longitudinal immune monitoring of patients. However, human immune repertoire contains public T cells, naïve T cells, and memory/effector T cells specific to diverse antigens, and this complexity adds to the challenges conventional systems are unable to solve (e.g., to identify cancer-associated T cells in the TCR-seq data).

Previous studies on the TCR repertoires of cancer patients reported that simple statistics, such as diversity and clonality, are associated with clinical outcome under certain conditions, substantiating the utilities of repertoire data as a potential prognostic factor. However, with the fast advancement of immunotherapies and rapid accumulation of TCR-seq data, more computational tools are required to bridge the gap between basic immunogenomics research and clinical applications beneficial to patients.

The disclosed systems and methods provide these needed tools through a novel framework executing ensemble machine learning software (referred to as TCRboost) that provides for de novo prediction of disease-associated immune repertoires using the TCR-seq data. Grouping of similar TCR sequences implicates shared antigen-specificity, and can be used to discover novel therapeutical targets. Conventional methods suffer from high computational expenses that cannot scale up to the magnitude of immune repertoire datasets.

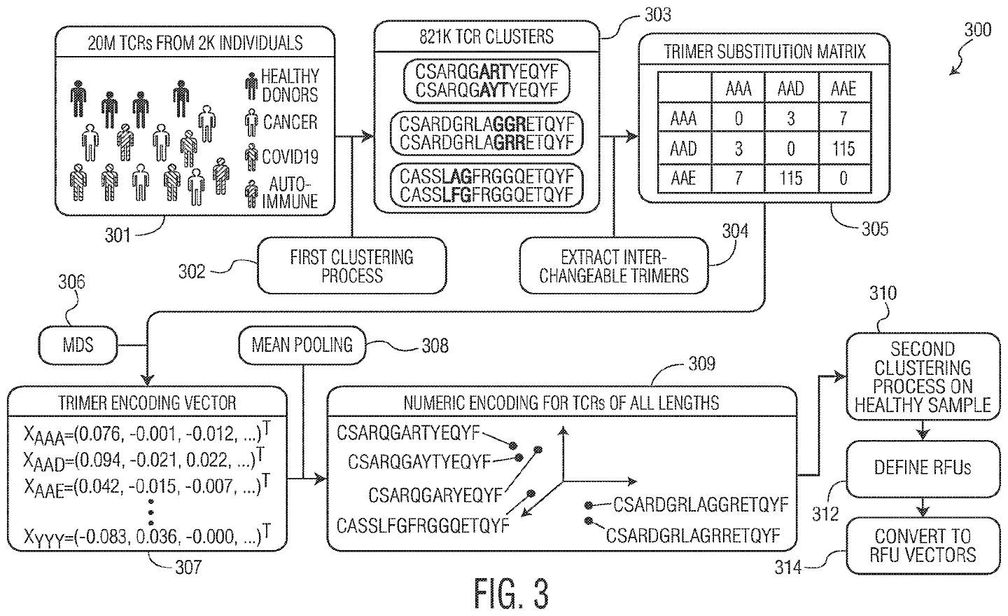

Referring now to FIG. 3, a flowchart illustrating the workflow of numeric encoding framework used in the RFU process 300 is shown. The RFU process 300 may begin by performing a first clustering process 302 on a sample TCR repertoire sequencing dataset 301. Any amount and type of sample TCR repertoire sequencing datasets to fill out a trimer substitution matrix, described in detail below may be used. For example, the number of TCRs in the sample TCR repertoire sequencing dataset 301 may range from approximately 10 million to over 100 million. In general, the more TCRs used in the sample TCR repertoire sequencing dataset 301, the more accurate the resulting numeric encoding may be. It is contemplated that the sample TCR repertoire sequencing dataset 301 may contain hundreds of millions or even billions of TCRs.

In an example, each sample may be processed to ensure data quality. First, TCR clones without a defined variable gene (TRBV) may be removed. Second, CDR3 amino acid sequences containing non-productive characters, such as “*” or “_”, may be excluded. Third, clones may be ranked by their estimated frequencies from high to low, and a top amount (e.g., 10,000) or maximum number of remaining clones may be selected. Fourth, the format of the TRBV gene names may be modified to be consistent with IMGT (imgt.org) convention. Samples may be derived from peripheral blood.

In an example, the sample TCR repertoire sequencing dataset 301 may include TCRs from over 2,000 samples covering cancer, infectious diseases, autoimmune disorders, and healthy controls merged into one file with over 20 million TCRs. Ultra-large-scale TCR clustering may be performed using the geometric isometry based antigen-specific TCR alignment (GIANA) process described in related Patent Cooperation Treaty (PCT) App. Pub. No. WO 2022/271566 entitled “TCR-Repertoire Framework for Multiple Disease Diagnosis” and filed on Jun. 17, 2022. The full disclosure of this application is incorporated herein by reference.

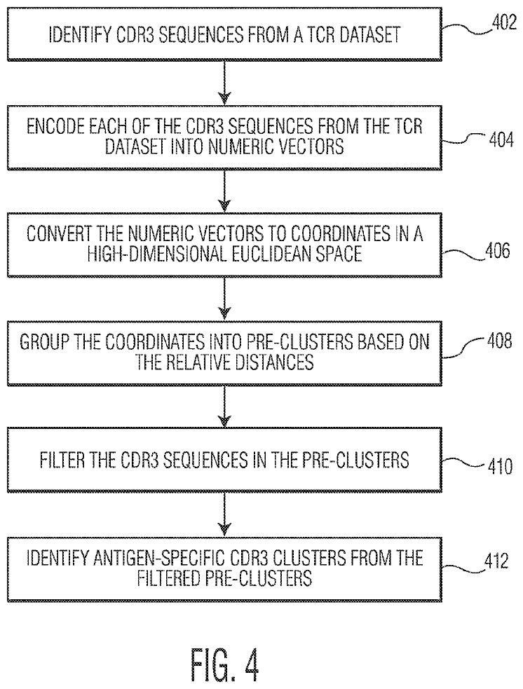

Referring now to FIG. 4, a flowchart of the GIANA process is shown. It should be noted that the steps shown in FIG. 4 may be performed by the TCR Engine 200, described above with reference to FIG. 2.

In step 402, the sample module 202 may identify CDR3 sequences from a TCR dataset. The sample module 202 may receive the TCR dataset from, for example, the database 107. In step 404, the encoding module 206 may encode each of the CDR3 sequences from the TCR dataset into numeric vectors. The numeric vectors may correspond to a sequence of amino acids in each of the CDR3 sequences.

In step 406, the conversion module 212 may convert the numeric vectors to coordinates in a high-dimensional Euclidean space. In step 408, the AI module 204 may generate a predictive model using a neural network. The neural network may learn to generate a tree data structure of the numeric vectors based on relative distances of the coordinates and may then group the coordinates into pre-clusters based on the relative distances. In step 410, the filtering module 208 may filter the CDR3 sequences in the pre-clusters. In step 412, the ID module 210 may identify antigen-specific CDR3 clusters from the filtered pre-clusters.

GIANA full mode (e.g., exact and variable gene included) maybe implemented to identify highly similar TCR clusters. The returned clusters may be processed in one or more ways. For example, clusters with more than 5 TCRs may be removed, as smaller clusters tend to have higher antigen specificity. Further, clusters with identical sequences may be removed.

The GIANA process may be used to close the gap between speed and prediction accuracy, with better precision and sensitivity than conventional methods (e.g., TCRdist) at approximately 600 times of its speed. GIANA may also allow ultrafast query of large reference cohorts, processing over 100 billion sequence comparisons within 3 minutes. In an example, GIANA may be able to compare 10+ TCRs against 107 reference sequences within 3 minutes. Applying GIANA to cluster large-scale TCR datasets may reveal novel insights of disease-specific receptors and provide a new solution to the repertoire classification task. Query of unseen TCR-seq samples against existing references using GIANA may achieve high accuracies and may be used to differentiate cancer, infectious disease, and autoimmune disorders. GIANA may be used as a TCR-based non-invasive multi-disease diagnostic platform.

Referring again to FIG. 3, in step 304, the resulting clusters 303 may be used to extract and define interchangeable trimers. In an example, an 8,000-by-8,000 trimer replacement matrix (M) with zeros 305 may be initialized. This matrix may be used to record a number of interchangeable trimer pairs calculated from the TCR clustering data. For each TCR cluster with s sequences, a position of a mismatched amino acid is marked as x. The flanking 1 positions of x: x−1, x, x+1 may be a trimer of interest. Given the high default alignment score cutoff (3.7) in GIANA, a majority of the small clusters (size≤5) may contain only 1 mismatch. If the cluster contains more than one mismatch, the above procedure may be iterated for each mismatch. This may allow each cluster to contribute s interchangeable trimers, designated as t₁, t₂ . . . tₛ. Notably, these trimers may be duplicated, and only unique ones may be kept. Each pair of trimers may add one to a corresponding entry in the trimer replacement matrix. For example, (t₁, t₂) may increase by 1 after a cluster is processed. Here, trimers in a parenthesis may indicate the location of the entry in the matrix M. After processing all the clusters, matrix M may be finalized.

The isometric embedding of trimers may derived by symmetrizing the trimer substitution matrix by:

$${M}_{s} = \frac{1}{2} ( M + {M}^{T} ) . \qquad ( Equation l )$$

This may be performed because the replacement of trimer pairs is not ordered. Next, the Pearson's correlation matrix of Mₛ, denoted as Pₛ, may be calculated. The (i, j) entry of Pₛ may be the Pearson's correlation of trimer i and j (trimers are ordered alphabetically). The Euclidean Distance Matrix (EDM) may be defined using the following formula:

$$EDM = \sqrt{1 - {P}_{s}} . \qquad ( Equation 2 )$$

In step 306, multi-dimensional scaling (MDS) may be applied to the EDM. In an example, dimensionality may be set to be 500. In another example, dimensionality may be incremented to 1,000, 1,500, and 2,000. In step 307, the outcome of this analysis may be a length-500 numeric vector (β) for each of the 8,000 amino acid trimers.

In step 308, mean pooling may be performed. In step 309, a process of numeric encoding of the CDR3 sequences may be performed. The numeric encoding process 309 may be able to incorporate TCRs with different lengths. Each CDR3 sequence may be stripped of the first two and last three amino acids (i.e., conserved motifs). The remaining sequence may be split into tiling trimers. For example, a sequence ASDTAGK may give ASD, SDT, DTA, TAG, and AGK. One or more n corresponding trimers may be selected and an average of the numeric vectors of the one or more n corresponding trimers may be used to obtain the numeric encoding vector with fixed dimensions of the TCR of interest:

$$x = \frac{1}{n} {\sum }_{i = 1}^{n} {β}_{i} . \qquad ( Equation 3 )$$

A key desirable feature of the numeric encoding of TCRs is antigen-specificity (i.e., TCRs specific to the same antigen(s) are expected to have closely located coordinates in the high-dimensional Euclidean space, where distance is well-defined). To evaluate the performance of this new approach, a dataset of T cells with experimentally solved antigen-specificities was used. First, antigens with fewer than 100 associated TCRs in the dataset were selected. This filter was applied because the TCRs reported from some high-throughput tetramer sorting experiments contained high rate of false positives. Next, 3,487 TCRs with unambiguously matched antigens were selected. The distances of each pair of TCRs were then calculated and used as predictors. The response vector was binary, being 1 when the TCRs in the pair specific to the same antigen, and 0 otherwise.

A receiver operator characteristic (ROC) curve was made to visualize the prediction accuracy, and an AUC of 0.59 was observed. Despite the low overall AUC, this approach reached a sensitivity of 19.5% at 95% specificity, which is the same level of previously described TCR clustering methods. This is due to the fact that neighboring TCRs share similar amino acid sequences, and similar TCRs may share antigen-specificity. Therefore, the new encoding method provides an absolute set of Euclidean coordinates that measures TCR similarity. Importantly, this embedding covers all the TCR lengths. The coordinate system has the quality of continuity: infinitesimally close TCRs are almost surely specific to the same antigen(s). This is simply because these TCRs will be “almost” identical. However, distal TCRs may also be specific to the same antigen(s), since it is repeatedly reported that TCRs with different motifs can recognize the same epitope. Therefore, it is expected that the sensitivity is low, but specificity is high.

After the numeric encoding process 309, a second clustering process 310 may be performed using the “points” in this space (each point is a TCR) to group them into antigen-specific clusters. The second clustering process 310 may be performed using a dataset composed of TCRs from healthy donors. In an example, the second clustering process 310 may be different than the first clustering process 302 described above. The second clustering process 310 may cluster sequences with different lengths. The second clustering process 310 may use a novel encoding approach, which may be derived from a large TCR dataset, and may carry antigen-specificity information.

In an example, the dataset of TCRs from healthy donors may include approximately 500,000 TCRs, although larger numbers are contemplated. For example, the number of TCRs in the dataset from healthy donors may range from approximately 500,000 to over 1 million. In general, the more TCRs used in the dataset from healthy donors, the more accurate the resulting clustering may be. It is contemplated that the dataset from healthy donors may contain hundreds of millions or even billions of TCRs. unsupervised k-means may be implemented in the 500 dimensional space, with, for example, 5,000 pre-defined centers (although any number may be chosen). The TCRs may be divided into 5,000 clusters because the top 10,000 abundant clones in each repertoire may cover most of the expanded TCRs. To ensure enough hits in each cluster in the downstream analysis, the number of clusters may not be very large. In an example, the average silhouette width reached 0.28, suggesting that the TCRs within each cluster were closer to the cluster centroid rather than other clusters (i.e., clustering was tight. This result suggests that although TCRs in the human immune repertoire display high diversity, they also show conserved distribution patterns in the high-dimensional encoding space, potentially related to the common antigenic challenges from the environment across different individuals.

In step 312, RFUs may be defined. In an example, the distribution of this distance was measured and it was observed that the 99% of the distances between TCRs and their cluster centroids were below 0.25, which is the cut-off of 95% specificity in the ROC curve. Therefore, it may be concluded that TCRs within the same cluster are mostly specific to the same antigens. This result suggested that the centroid of each TCR cluster can be viewed as a “functional unit” of an immune repertoire (i.e., an RFU), with each unit covering a spectrum of antigens. The immune repertoire may be viewed as patches of such units to cover all the possible pathogens to be encountered during lifetime. The number of antigens that each unit responds to may be very large, considering the enormous amount of internal and external immune challenges human body will receive.

In step 314, TCR repertoire samples may be converted into RFU vectors. For a given TCR repertoire sample, the 500-dimensional encoding vector may be calculated for each TCR. The vector may then be compared to each of the 5,000 RFU centroid using Spearman's correlation. The vector may be designated to the RFU with the highest correlation. The rank correlation may be used instead of Euclidean distance to assign the RFUs to reduce the impact of outlier coordinates and to accelerate computational speed. In an example, over 70% of the TCRs in a sample had Spearman's correlation greater than 0.6, suggesting that most TCRs can be assigned to an RFU with similar centroid. Processing all K TCRs in a sample may result in a length-5,000 vector, with each entry the count of TCRs assigned to the related RFU. The final vector may be the vector normalized by K and multiplied by 10,000. The numeric encoding derived from the trimer replacement matrix may allow for an alternative way to visualize and compare repertoire samples.

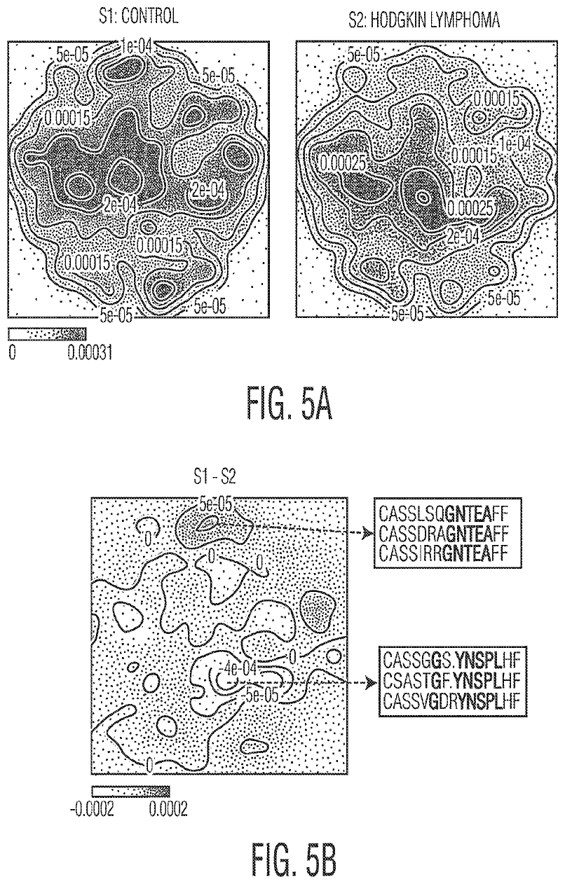

Referring now to FIGS. 5A-5B, diagrams illustrating repertoire visualization using the trimer numeric encoding of TCRs are shown. FIG. 5A shows a 2-dimensional density plot of t-distributed stochastic neighbor embedding (t-SNE) coordinates calculated from the original 500-dimensional numeric encoding matrix for both control and a Hodgkin lymphoma patient. FIG. 5B shows a difference in the density by subtracting control density with lymphoma patients. Selected regions of the density plot are highlighted to show enriched TCR motifs.

To illustrate contrast visualization of immune repertoire samples, two samples from a recent study on B cell Hodgkin lymphoma were selected, one from the healthy control and the other lymphoma patient. Both samples were derived from the peripheral blood. After numeric transformation, each sample was converted into a 10,000-by-500 matrix, with each row corresponding to a TCR, and column a dimension in the encoding space. The distribution of the TCRs in this space was then visualized using tSNE as the dimension reduction technique. Other methods, such as UMAP, or PCA may also serve the same purpose. Each TCR may be represented by a point on the 2-D tSNE plot shown in FIG. 5A, from which the different patterns of TCR distributions between HC and lymphoma patient can be readily visualized. A differential density plot shown in FIG. 5B may be generated by subtracting the density of lymphoma patient from the control, and observing regions (mapped to TCR motifs) showing selective enrichment in lymphoma (YNSPL) or in the HC (GNTEA). This analysis illustrates an example of how the numeric encoding approach can lead to differentially regulated TCR patterns in the cancer patients. As described above, the RFU process may be able to diagnose cancer from a general population, even with other factors present (e.g., herd immunity from COVID-19).

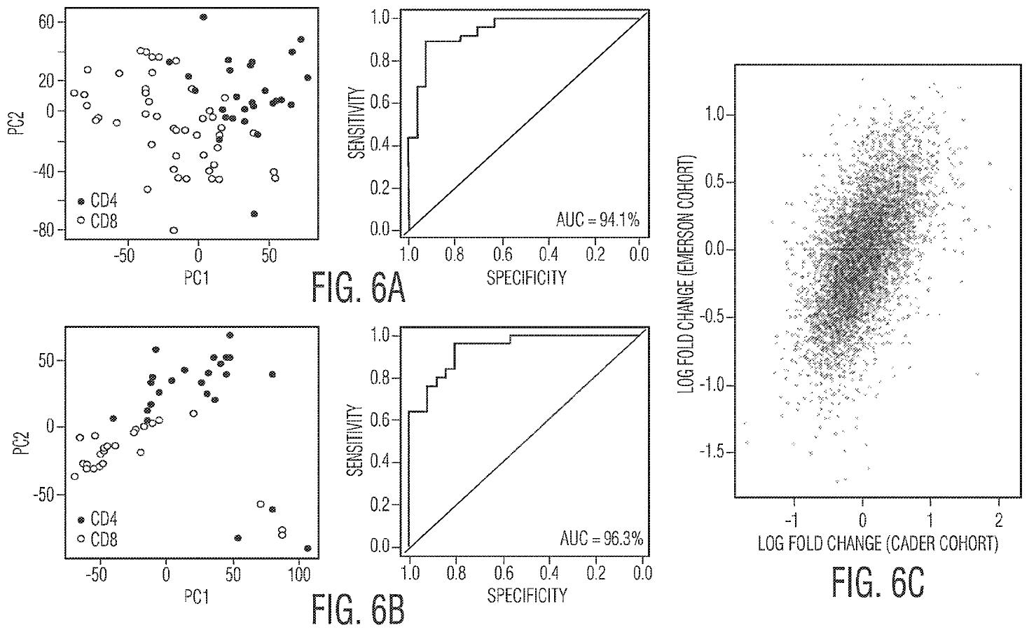

Referring now to FIGS. 6A-6C, charts illustrating how the RFU process differentiates CD4 and CD8 T-cell repertoires are shown. CD8+ and CD4+ T-cells recognize different antigens bounded by MHC class-I and class-II respectively. Due to the differences between class-I and II epitopes, it is expected that their specific T-cell receptors may carry distinct features. In support of this view, a recent study reported a significant statistical co-occurrence of certain TCR variable gene and MHC alleles. Another study also reported different signatures between CD8 and CD4 T-cells, though the features were defined at the repertoire level. As RFUs are expected to be antigen-specific, we hypothesized that at least a subset of RFUs are consistently CD4+ or CD8+.

Two datasets containing both CD4+ and CD8+ T cell repertoires were processed. FIG. 6A illustrates a sample containing 27 CD8+ and 46 CD4+ TCR repertoire samples, all from lymphoma patients. FIG. 6B illustrates a sample containing 25 CD8+ and 25 CD4+ samples, all from healthy donors. RFU transformation was performed for both datasets, followed by PCA analysis. Both PC1 and PC2 were driven by the differences between CD4+ and CD8+ T cells. Using the first PCs as a predictor, RFU can almost perfectly separate CD4 from CD8 repertoires. This result indicated that despite antigen diversity, a large fraction of RFUs preserved the CD4+ or CD8+ identity across different individuals. To prove this hypothesis, differential RFU analysis was performed by conducting a two-sample Wilcoxon rank test between CD4 and CD8 groups for each RFU, and the mean fold change was estimated. As expected, most RFUs showed consistent fold changes from the two sample cohorts (p≤2.2e-16, Spearman's correlation test), which supported the conclusion above. FIG. 6C shows a scatter plot showing high correlations between the fold changes of the two cohorts. Fold change for each RFU was calculated as the mean value of CD4 samples over the mean value of the CD8 samples.

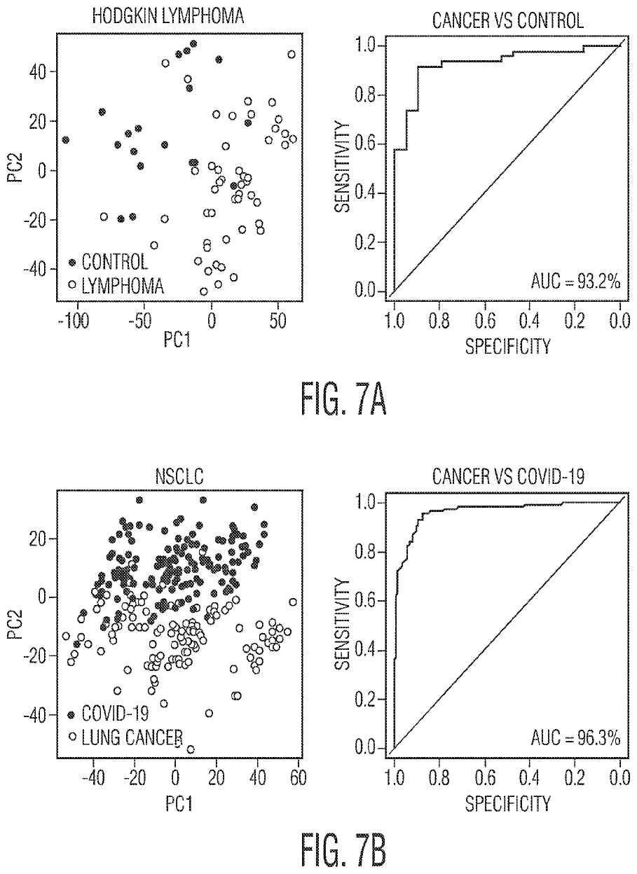

Referring to FIGS. 7A-7B, charts illustrating how the RFU process differentiates cancer patients from healthy controls (“HCs”) and COVID-19 patients are shown. In an example, samples containing 19 HCs and 56 lymphoma patients processed in the same cohort were analyzed. Each sample was converted into an RFU vector as described above. Each dataset was represented as a matrix with 5,000 rows, each for one RFU, and N columns, with N being the sample size. Principal component analysis (PCA) was performed on both datasets. The first two PCs are shown in FIG. 7A. This simple, unsupervised approach was readily sufficient to differentiate cancer from non-cancer subjects. Using PC1+PC2 as a predictor, it reached an AUC of 93.2%. This result suggested that cancer is a major immunologic disease that drives the variations in the structure of TCR repertoires, which can be captured through our RFU approach.

As COVID-19 infection has affected over 16% of Americans, it is expected that a marked proportion of the “healthy” population will be COVID-19 patients. To test if this new situation affects cancer prediction by a new dataset consisting of 121 non-small cell lung cancer (“NSCLC”) patients and 160 individuals recently infected with COVID-19 was built. Both datasets were profiled with the latest version of reagents by the immunoSEQ platform of AdaptiveBiotech. Using PCA on the RFU matrix, clear separation of the lung cancer group from the COVID-19 patients was observed, though the cancer vs non-cancer difference is only driving PC2, with AUC reaching 96.3% as shown in FIG. 7B. This result indicates that the immunologic responses caused by malignant lung tumors is different from acute virus infections in the upper respiratory track, which can be captured using the RFU analysis. Notably, the RFU method provides a uniform feature set that can be used by future supervised machine learning methods, such as boosting, SVM, random forest, deep neural network models, etc. It is expected to that these methods will reach higher prediction accuracy and consistency with sufficient training samples for the task of cancer/non-cancer classification.

For the purposes of this disclosure a module is a software, hardware, or firmware (or combinations thereof) system, process or functionality, or component thereof, that performs or facilitates the processes, features, and/or functions described herein (with or without human interaction or augmentation). A module may include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing examples. In other words, functional elements being performed by single or multiple components, in various combinations of hardware and software or firmware, and individual functions, may be distributed among software applications at either the client level or server level or both. In this regard, any number of the features of the different examples described herein may be combined into single or multiple examples, and alternate examples having fewer than, or more than, all of the features described herein are possible.

Functionality may also be, in whole or in part, distributed among multiple components, in manners now known or to become known. Thus, a myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the examples of methods presented and described as flowcharts in this disclosure are provided by way of example in order to provide a more complete understanding of the technology. The disclosed methods are not limited to the operations and logical flow presented herein. Alternative examples are contemplated in which the order of the various operations is altered and in which sub-operations described as being part of a larger operation are performed independently.

While various examples have been described for purposes of this disclosure, such examples should not be deemed to limit the teaching of this disclosure to those examples. Various changes and modifications may be made to the elements and operations described above to obtain a result that remains within the scope of the systems and processes described in this disclosure.

What is claimed is:

1. A method comprising:
encoding short peptide sequences of T-cell receptors (TCRs) in a large sequence of TCR samples into a numeric vector with fixed dimensions, the short peptide sequences having different lengths;
transforming each TCR in a pool of existing TCRs from healthy individuals into the numeric vector to generate a distribution of the numeric vector in a high-dimensional Euclidean space;
clustering the transformed TCRs from the pool of existing TCRs into antigen-specific clusters, wherein a centroid of each cluster is a repertoire functional unit (“RFU”);
transforming each TCR in one or more TCR repertoire samples into the numeric vector;
assigning one or more of the transformed TCRs from each of the one or more TCR repertoire samples to an RFU based on one or more correlations; and
normalizing a number of RFUs with assigned transformed TCRs from the each of the one or more TCR repertoire samples by a total number of the TCRs in a corresponding TCR repertoire sample to generate a fixed-length RFU vector for each of the one or more TCR repertoire samples.

2. A method comprising:
encoding short peptide sequences of T-cell receptors (TCRs) in a large sequence of TCR samples into a numeric vector with fixed dimensions, the short peptide sequences having different lengths, wherein the encoding comprises:
performing ultra-large-scale TCR clustering of the TCRs in the large sequence;
defining interchangeable trimers using the clustered TCRs;
deriving an isometric embedding of the interchangeable trimers;
defining a Euclidean Distance Matrix (EDM) representing the interchangeable trimers;
applying Multi-Dimensional Scaling (MDS) to the EDM to derive a numeric vector for each of the interchangeable trimers;
removing one or more amino acids from each complementarity-determining region 3 (CDR3) of each clustered TCR to form a respective sequence;
splitting each respective sequence into tiling trimers;
selecting one or more corresponding interchangeable trimers of each clustered TCR; and
averaging the numeric vector of the one or more corresponding interchangeable trimers to obtain the numeric vector with fixed dimensions;
transforming each TCR in a pool of existing TCRs from healthy individuals into the numeric vector to generate a distribution of the numeric vector in a high-dimensional Euclidean space;
clustering the transformed TCRs from the pool of existing TCRs into antigen-specific clusters, wherein a centroid of each cluster is a repertoire functional unit (“RFU”);
transforming each TCR in one or more TCR repertoire samples into the numeric vector;
assigning one or more of the transformed TCRs from each of the one or more TCR repertoire samples to an RFU based on one or more correlations;
normalizing a number of RFUs with assigned transformed TCRs from the each of the one or more TCR repertoire samples by a total number of the TCRs in a corresponding TCR repertoire sample to generate a fixed-length RFU vector for each of the one or more TCR repertoire samples;
performing a first principal component (PC1) analysis on the fixed-length RFU vectors of the one or more TCR repertoire samples;
performing a second principal component (PC2) analysis on the fixed-length RFU vectors of the one or more TCR repertoire samples; and
comparing results of the PC1 analysis against results of the PC2 analysis to distinguish between a set of healthy samples and a set of non-healthy samples.

3. The method of claim 2, wherein the distinguishing between the set of healthy samples and a set of non-healthy samples achieves an area under the curve (AUC) of at least 93%.

4. The method of claim 1, where in the fixed-length RFU vector represents a relative abundance of a given RFU in a respective TCR repertoire sample.

5. The method of claim 1, wherein the large sequence comprises a dataset with a number of TCRs ranging from approximately 10 million to 100 million.

6. The method of claim 1, wherein the large sequence comprises TCRs covering one or more of healthy donors, cancer, infectious diseases, and autoimmune disorders.

7. The method of claim 1, wherein the encoding the short peptide sequences comprises:
performing ultra-large-scale TCR clustering of the TCRs in the large sequence;
defining interchangeable trimers using the clustered TCRs;
deriving an isometric embedding of the interchangeable trimers;
defining a Euclidean Distance Matrix (EDM) representing the interchangeable trimers;
applying Multi-Dimensional Scaling (MDS) to the EDM to derive a numeric vector for each of the interchangeable trimers;
removing one or more amino acids from each complementarity-determining region 3 (CDR3) of each clustered TCR to form a respective sequence;
splitting each respective sequence into tiling trimers;
selecting one or more corresponding interchangeable trimers of each clustered TCR; and
averaging the numeric vector of the one or more corresponding interchangeable trimers to obtain the numeric vector with fixed dimensions of each clustered TCR.

8. The method of claim 1, wherein the pool of existing TCRs from healthy individuals comprises a dataset with a number of TCRs ranging from approximately 500,000 to approximately 1 million.

9. The method of claim 1, wherein the one or more correlations comprise a rank correlation.

10. The method of claim 1, wherein the one or more TCR repertoire samples comprise a first set of samples and a second set of samples.

11. The method of claim 10, further comprising:
performing a first principal component (PC1) analysis on the fixed-length RFU vectors of the one or more TCR repertoire samples;
performing a second principal component (PC2) analysis on the fixed-length RFU vectors of the one or more TCR repertoire samples; and
comparing results of the PC1 analysis against results of the PC2 analysis to distinguish between the first set of samples and the second set of samples.

12. The method of claim 11, wherein the first set of samples comprise healthy controls and the second set of samples comprise one or more of cancer, infectious diseases, and autoimmune disorders.

13. The method of claim 11 or 12, wherein the distinguishing between the first set of samples and the second set of samples achieves an area under the curve (AUC) of at least 90%.

14. The method of any one of claims 11-13, wherein the distinguishing between the first set of samples and the second set of samples achieves an area under the curve (AUC) of at least 93%.

15. The method of any one of claims 11-14, wherein the distinguishing between the first set of samples and the second set of samples achieves an area under the curve (AUC) of at least 94%.

16. The method of any one of claims 11-15, wherein the distinguishing between the first set of samples and the second set of samples achieves an area under the curve (AUC) of at least 96%.

17. A computing device comprising:
a processor operatively coupled to a memory storing non-transitory computer-readable instructions that, when executed by the processor, cause the processor to:
encode short peptide sequences of T-cell receptors (TCRs) in a large sequence of TCR samples into a numeric vector with fixed dimensions, the short peptide sequences having different lengths;
transform each TCR in a pool of existing TCRs from healthy individuals into the numeric vector to generate a distribution of the numeric vector in a high-dimensional Euclidean space;
cluster the transformed TCRs from the pool of existing TCRs into antigen-specific clusters, wherein a centroid of each cluster is a repertoire functional unit (“RFU”);
transform each TCR in one or more TCR repertoire samples into the numeric vector;
assign one or more of the transformed TCRs from each of the one or more TCR repertoire samples to an RFU based on one or more correlations; and
normalize a number of RFUs with assigned transformed TCRs from the each of the one or more TCR repertoire samples by a total number of the TCRs in a corresponding TCR repertoire sample to generate a fixed-length RFU vector for each of the one or more TCR repertoire samples.

18. The computing device of claim 17, where in the fixed-length RFU vector represents a relative abundance of a given RFU in a respective TCR repertoire sample.

19. The computing device of claim 17, wherein the large sequence comprises a dataset with a number of TCRs ranging from approximately 10 million to 100 million.

20. The computing device of claim 17, wherein the large sequence comprises TCRs covering one or more of healthy donors, cancer, infectious diseases, and autoimmune disorders.

21. The computing device of claim 17, wherein the encoding the short peptide sequences comprises:
performing ultra-large-scale TCR clustering of the TCRs in the large sequence;
defining interchangeable trimers using the clustered TCRs;
deriving an isometric embedding of the interchangeable trimers;
defining a Euclidean Distance Matrix (EDM) representing the interchangeable trimers;
applying Multi-Dimensional Scaling (MDS) to the EDM to derive a numeric vector for each of the interchangeable trimers;
removing one or more amino acids from each complementarity-determining region 3 (CDR3) of each clustered TCR to form a respective sequence;
splitting each respective sequence into tiling trimers;
selecting one or more corresponding interchangeable trimers of each clustered TCR; and
averaging the numeric vector of the one or more corresponding interchangeable trimers to obtain the numeric vector with fixed dimensions of each clustered TCR.

22. The computing device of claim 17, wherein the pool of existing TCRs from healthy individuals comprises a dataset with a number of TCRs ranging from approximately 500,000 to approximately 1 million.

23. The computing device of claim 17, wherein the one or more correlations comprise a rank correlation.

24. The computing device of claim 17, wherein the one or more TCR repertoire samples comprise a first set of samples and a second set of samples.

25. The computing device of claim 24, wherein the non-transitory computer-readable instructions that, when executed by the processor, further cause the processor to:
perform a first principal component (PC1) analysis on the fixed-length RFU vectors of the one or more TCR repertoire samples;
perform a second principal component (PC2) analysis on the fixed-length RFU vectors of the one or more TCR repertoire samples; and
compare results of the PC1 analysis against results of the PC2 analysis to distinguish between the first set of samples and the second set of samples.

26. The computing device of claim 25, wherein the first set of samples comprise healthy controls and the second set of samples comprise one or more of cancer, infectious diseases, and autoimmune disorders.

27. The computing device of claim 25 or 26, wherein the non-transitory computer-readable instructions that cause the processor to distinguish between the first set of samples and the second set of samples achieve an area under the curve (AUC) of at least 90%.

28. The computing device of any one of claims 25-27, wherein the distinguishing between the first set of samples and the second set of samples achieves an area under the curve (AUC) of at least 93%.

29. The computing device of any one of claims 25-28, wherein the distinguishing between the first set of samples and the second set of samples achieves an area under the curve (AUC) of at least 94%.

30. The computing device of any one of claims 25-29, wherein the distinguishing between the first set of samples and the second set of samples achieves an area under the curve (AUC) of at least 96%.

31. A non-transitory computer-readable storage medium tangibly encoded with computer-executable instructions, that when executed by a processor associated with a computing device, cause the processor to:
encode short peptide sequences of T-cell receptors (TCRs) in a large sequence of TCR samples into a numeric vector with fixed dimensions, the short peptide sequences having different lengths;
transform each TCR in a pool of existing TCRs from healthy individuals into the numeric vector to generate a distribution of the numeric vector in a high-dimensional Euclidean space;
cluster the transformed TCRs from the pool of existing TCRs into antigen-specific clusters, wherein a centroid of each cluster is a repertoire functional unit (“RFU”);
transform each TCR in one or more TCR repertoire samples into the numeric vector;
assign one or more of the transformed TCRs from each of the one or more TCR repertoire samples to an RFU based on one or more correlations; and
normalize a number of RFUs with assigned transformed TCRs from the each of the one or more TCR repertoire samples by a total number of the TCRs in a corresponding TCR repertoire sample to generate a fixed-length RFU vector for each of the one or more TCR repertoire samples.

32. The non-transitory computer-readable storage medium of claim 31, where in the fixed-length RFU vector represents a relative abundance of a given RFU in a respective TCR repertoire sample.

33. The non-transitory computer-readable storage medium of claim 31, wherein the large sequence comprises a dataset with a number of TCRs ranging from approximately 10 million to 100 million.

34. The non-transitory computer-readable storage medium of claim 31, wherein the large sequence comprises TCRs covering one or more of healthy donors, cancer, infectious diseases, and autoimmune disorders.

35. The non-transitory computer-readable storage medium of claim 31, wherein the encoding the short peptide sequences comprises:
performing ultra-large-scale TCR clustering of the TCRs in the large sequence;
defining interchangeable trimers using the clustered TCRs;
deriving an isometric embedding of the interchangeable trimers;
defining a Euclidean Distance Matrix (EDM) representing the interchangeable trimers;
applying Multi-Dimensional Scaling (MDS) to the EDM to derive a numeric vector for each of the interchangeable trimers;
removing one or more amino acids from each complementarity-determining region 3 (CDR3) of each clustered TCR to form a respective sequence;
splitting each respective sequence into tiling trimers;
selecting one or more corresponding interchangeable trimers of each clustered TCR; and
averaging the numeric vector of the one or more corresponding interchangeable trimers to obtain the numeric vector with fixed dimensions of each clustered TCR.

36. The non-transitory computer-readable storage medium of claim 31, wherein the pool of existing TCRs from healthy individuals comprises a dataset with a number of TCRs ranging from approximately 500,000 to approximately 1 million.

37. The non-transitory computer-readable storage medium of claim 31, wherein the one or more correlations comprise a rank correlation.

38. The non-transitory computer-readable storage medium of claim 31, wherein the one or more TCR repertoire samples comprise a first set of samples and a second set of samples.

39. The non-transitory computer-readable storage medium of claim 38, wherein the non-transitory computer-readable instructions that, when executed by the processor, further cause the processor to:
perform a first principal component (PC1) analysis on the fixed-length RFU vectors of the one or more TCR repertoire samples;
perform a second principal component (PC2) analysis on the fixed-length RFU vectors of the one or more TCR repertoire samples; and
compare results of the PC1 analysis against results of the PC2 analysis to distinguish between the first set of samples and the second set of samples.

40. The non-transitory computer-readable storage medium of claim 39, wherein the first set of samples comprise healthy controls and the second set of samples comprise one or more of cancer, infectious diseases, and autoimmune disorders.

41. The non-transitory computer readable medium of claim 39 or 40, wherein the computer-executable instructions that cause the processor to distinguish between the first set of samples and the second set of samples achieve an area under the curve (AUC) of at least 90%.

42. The non-transitory computer readable medium of any one of claims 39-41, wherein the distinguishing between the first set of samples and the second set of samples achieves an area under the curve (AUC) of at least 93%.

43. The non-transitory computer readable medium of any one of claims 39-42, wherein the distinguishing between the first set of samples the second set of samples achieves an area under the curve (AUC) of at least 94%.

44. The non-transitory computer readable medium of any one of claims 39-42, wherein the distinguishing between the first set of samples and the second set of samples achieves an area under the curve (AUC) of at least 96%.
